(19) United States
(12) Patent Application Publication
(10) Pub. No.: US 2017/0042980 A1
(43) Pub. Date: Feb. 16, 2017

(54) Degradation of Beta-Amyloid Proteins With Keratinase

(71) Applicant: Kirk Steven Johnson, Ewa Beach, HI (US)

(72) Inventor: Kirk Steven Johnson, Ewa Beach, HI (US)

(21) Appl. No.: 15/339,093

(22) Filed: Oct. 31, 2016

(51) Int. Cl. A61K 38/48; A61K 9/12; C07K 16/28; A61K 9/00

(52) U.S. Cl. CPC A61K 38/48; A61K 9/0043; A61K 9/12; C07K 16/28; C07K 2317/569; C07K 2319/95; C07K 2319/74

(57) ABSTRACT

The present invention involves using a keratinase enzyme to degrade beta-amyloid proteins and plaques related to Alzheimer's disease.

### BACKGROUND

The present invention relates to degrading beta-amyloid fibrils associated with Alzheimer's disease in order to help slow or stop the progression of the disease. In Alzheimer's it is believed that plaques formed by beta-amyloid fibrils cause severe damage and leads to the progression of the dementia.

Keratinase has been shown to degrade prions (Enzymatic Degradation of Prion Protein in Brain Stem from Infected Cattle and Sheep, Dr. Jan P. M. Langeveld), which is an amyloid type protein, and it degrades keratin, which is also an amyloid protein.

By using keratinase to degrade beta-amyloid fibrils that form the plaques associated with the disease the hope is to slow or stop the negative effects of the dementia and to allow people to live out the rest of their life with out as much drastic consequences that normally occurs.

### References

1. Langeveld, Jan P., Dick F. Van De Wiel, Giles C. Shih, G. Garssen, Alex Bossers, and Jason C. Shih. “The Journal of Infectious Diseases.” Enzymatic Degradation of Prion Protein in Brain Stem from Infected Cattle and Sheep. The Journal of Infectious Diseases, 1 Aug. 2016. Web. 7 Jul. 2016.
2. Van Ginkel, F. W., R. J. Jackson, Y. Yuki, and J. R. McGhee. “Cutting Edge: The Mucosal Adjuvant Cholera Toxin Redirects Vaccine Proteins into Olfactory Tissues.” National Center for Biotechnology Information. U.S. National Library of Medicine, 1 Nov. 2000. Web. 7 Jul. 2016.

### SUMMARY

This invention involves using keratinase enzyme to degrade beta-amyloid plaques associated with Alzheimer's disease and the methods and clams outline ways of getting the enzyme into the brain as a treatment. Whether it is attached as a chimeric protein, to either the transferrin protein or a single chain variable fragment, or coupled to either of these, or in a nasal mucosal adjuvant, these are all just potential ways to get the enzyme into the brain for treatment.

### DESCRIPTION

### Detailed Methods of Use

Crossing the blood brain barrier is always a difficult hurdle with neurological treatments. The transferrin protein is a protein that delivers iron to cells in the brain and the transferrin receptor is what it binds to the transferrin receptor to be taken across the blood brain barrier. By attaching the enzyme to the transferrin protein the enzyme could then enter the brain and degrade the amyloid fibrils.

Single chain variable fragments are just the variable region of an antibody. By using only this fragment you lower the immunogenicity of the protein, since most of the immunogenicity occurs as a result of the constant region of an antibody. The idea again would be to attach the enzyme to a single chain variable fragment that had an affinity for the transferrin receptor in order to cross the blood brain barrier and treat a patient. Some common methods of attaching two proteins are either coupling them together or creating a chimeric protein using a vector like yeast or bacteria to produce the protein. If using a vector yeast would probably be the better option since it is Eukaryotic and would then glycosylate the protein giving it a lower immunogenicity.

Nasal mucosal adjuvants have been shown to allow proteins passive entrance to the brain through the olfactory nerve (Cutting Edge: The Mucosal Adjuvant Cholera Toxin Redirects Vaccine Proteins into Olfactory Tissues, Fredrick W. van Ginkel). The idea here would be to exploit this by including keratinase in the adjuvant in order to allow it to access the brain.

What is claimed:

1. By treating patients diagnosed with Alzheimer's disease with the keratinase enzyme the plaques caused by the beta-amyloid fibers will degrade and this will slow progression of disease and help prevent patient from negative consequences of the dementia. Creating a bio-medical product with the enzyme and attaching it to something that can cross the blood brain barrier can accomplish this. Examples of this are:
Coupling or creating a chimeric protein with the enzyme attached to a single chain variable fragment with a low affinity binding to the Transferrin receptor
Administering the enzyme in a nasal mucosa or aerosol for passive entry into the brain along the olfactory nerve.
